(19) United States
(12) Patent Application Publication
(10) Pub. No.: US 2020/0078395 A1
(43) Pub. Date: Mar. 12, 2020

(54) AVERTING COMPLICATIONS OF PUMP INHIBITOR THERAPY BY EFFERVESCENT CALCIUM MAGNESIUM CITRATE

(71) Applicant: THE BOARD OF REGENTS OF THE UNIVERSITY OF TEXAS SYSTEM, Austin, TX (US)

(72) Inventors: Charles Y.C. PAK, Dallas, TX (US); Khashayar SAKHAEE, Dallas, TX (US); Orson W. MOE, Dallas, TX (US)

(21) Appl. No.: 16/563,439

(22) Filed: Sep. 6, 2019

Related U.S. Application Data
(60) Provisional application No. 62/728,100, filed on Sep. 7, 2018.

(51) Int. Cl. A61K 33/06; A61K 31/194; A61K 9/00; A61K 9/08; A61K 9/46; A61P 13/00

(52) U.S. Cl. CPC A61K 33/06; A61K 31/194; A61K 9/0095; A61K 45/06; A61K 9/0007; A61P 13/00; A61K 9/08

(57) ABSTRACT

Disclosed is the use of an effervescent preparation of calcium and magnesium with additional citric acid in a defined ratio to reduce serum C-carboxy-terminal telopeptide (CTX), increase urinary Mg, increase FEMg, increase urinary citrate, reduce urinary ammonium, and decrease putative serum fibroblast growth factor 23 (FGF23) in subjects taking proton pump inhibitors.

### CROSS-REFERENCE TO RELATED APPLICATIONS

This application claims the benefit of priority to U.S. Provisional Patent Application Ser. No. 62/728,100, filed Sep. 7, 2018, the entire contents of which are hereby incorporated by reference in their entirety.

### BACKGROUND OF THE INVENTION

### 1. Field of the Invention

The present invention relates generally to the fields of biology, chemistry, and medicine. More particularly, it concerns methods of reducing serum C-carboxy-terminal telopeptide (CTX), increasing serum and urinary Mg, increasing fractional excretion of magnesium (FEMg), increasing urinary citrate and reducing urinary ammonium, and decreasing putative serum fibroblast growth factor 23 (FGF23) in patients receiving or prescribed a proton pump inhibitor (PPI) therapy.

### 2. Background

PPIs are effective inhibitors of gastric acid secretion. Thus, they are widely used for the control of gastric ulcer-gastritis, erosive esophagitis (gastroesophageal reflux disease), peptic ulcer disease (duodenal ulcer), and heartburn. PPIs are among the most commonly used drugs worldwide, with annual sales in the USA alone exceeding $10 billion.

However, several population-based studies have reported increased risk of skeletal fractures during prolonged PPI use (Roux, 2009). Moreover, hypomagnesemia has been reported during PPI treatment (Kuipers, 2009). The symptomatic presentation of some of the cases suggested that magnesium deficiency with tissue depletion of magnesium had occurred. The alert by the FDA of these complications led to their inclusion under Warnings & Precautions in the package inserts of PPIs (FDA, 2011; Tamura, 2012). More recently, PPI use has been associated with increased risk of chronic kidney disease (CKD), defined by estimated glomerular filtration rate (eGFR) of less than 60 ml/min (Wijarnpreecha, 2017; Xie, 2017). These reports led to a news alert by the leading international nephrology society (ASN, 2017).

The exact pathogenic mechanism for the aforementioned complications from PPI use is unclear. For the first two complications (osteoporosis and hypomagnesemia), it is suspected that the induction by PPI of hypo- or achlorhydria impairs the solubility of calcium and magnesium salts, impairing calcium and magnesium absorption (Pak, 1989). There is as yet no explanation for the development of third complication of PPI—increased risk of CKD.

Currently, there are no specific guidelines for the prevention or treatment of bone loss and fragility fractures (osteoporosis), hypomagnesemia (magnesium deficiency), and increased incidence of CKD from PPI therapy. Despite potential of calcium-magnesium formulations to avert above disturbances, currently available calcium-magnesium preparations are tablet formulations or emulsions with limited solubility in states of impaired gastric acid secretion from PPI. There is clearly a need for a calcium-magnesium preparation that delivers soluble calcium and magnesium during PPI.

### SUMMARY OF THE INVENTION

The inventors have surprisingly determined that a composition comprising calcium and magnesium in a soluble form (“EffCaMgCit”) reduced serum CTX, increased serum and urinary Mg, increased FEMg, increased urinary citrate, reduced urinary ammonium, and decreased putative serum FGF23 in patients receiving or prescribed a PPI therapy.

In some aspects, disclosed is a method of reducing serum CTX in a patient, the method comprising administrating a composition comprising calcium, magnesium, and citrate excess, wherein serum CTX is reduced. In some aspects, bone loss is decreased.

In some aspects, disclosed is a method of increasing serum and urinary Mg in a patient, the method comprising administrating a composition comprising calcium, magnesium, and well-defined citrate excess, wherein the serum and urinary Mg are increased. In some aspects, tissue magnesium stores are increased. In some aspects, the patient has or is at risk of having CKD.

In some aspects, disclosed is a method of increasing FEMg in a patient, the method comprising administrating a composition comprising calcium, magnesium, and citrate excess, wherein the FEMg is increased. In some aspects, the patient has or is at risk of having CKD.

In some aspects, disclosed is a method of increasing urinary citrate and reducing urinary ammonium in a patient, the method comprising administrating a composition comprising calcium, magnesium, and citrate excess, wherein the urinary citrate is increased and urinary ammonium is decreased. In some aspects, the acid load is increased in the patient. In some aspects, the patient has or is at risk of having CKD.

In some aspects, disclosed is a method of decreasing putative serum FGF23 in a patient, the method comprising administrating a composition comprising calcium, magnesium, and citrate excess, wherein the FGF23 is decreased. In some aspects, the patient has or is at risk of having CKD.

In some aspects, the composition is administered to a patient receiving or prescribed a PPI therapy.

Some aspects of this disclosure involve administering a composition comprising calcium, magnesium, and citrate. In some aspects, this composition is in a soluble form. In some aspects, the composition is a mixture. In some aspects, the mixture is in the form of a powder, to be dissolved in aqueous medium. In some aspects, the composition is an aqueous solution. In some aspects, the aqueous solution is prepared by dissolving the dose comprising the mixture in water. In some aspects, the solution elaborates carbon dioxide upon dissolution. In some aspects, the composition is mixed with a PPI.

In some aspects, the composition comprises an effective amount of calcium, magnesium, and citrate. In some embodiments, the composition comprises at least, at most, or exactly 100, 110, 120, 130, 140, 150, 160, 170, 180, 190, 200, 220, 240, 260, 280, 300, 350, 400, 450, 500, 520, 540, 560, 580, 600, 610, 620, 630, 640, 650, 660, 670, 680, 690, or 700 mg of calcium, or any range derivable therein. In some embodiments, the composition comprises at least, at most, or exactly 10, 20, 30, 40, 50, 60, 70, 80, 90, 100, 110, 120, 130, 140, 150, 160, 165, 170, 175, 180, 185, 190, 195, or 200 mg of magnesium, or any range derivable therein. In some embodiments, the composition comprises at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 meq of citrate excess, or any range derivable therein. In some embodiments, the composition comprises at least, at most, or exactly 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or meq of total soluble citrate, or any range derivable therein. In some embodiments, the molar ratio of calcium to citrate (expressed in millimoles) is at least, at most, or exactly 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.2, 1.4, 1.6, 1.8, or 2.0 (or any derivable range therein).

In some aspects, the composition comprises about 250 mg calcium to about 600 mg of calcium, from about 70 mg magnesium to about 175 mg of magnesium, at least 10 meq of citrate excess, and at least 20 meq of total soluble citrate, wherein the Ca/citrate molar ratio is from 0.4 to 0.7. In some aspects, the composition comprises 380 mg (19 meq) of calcium, 122 mg (10 meq) of magnesium, 21 meq of citrate excess, and 50 meq of total soluble citrate, wherein the Ca/citrate molar ratio is from 0.4 to 0.7. In some aspects, the Ca/citrate molar ratio is 0.57.

The composition may be administered in any suitable manner. For example, it may be administered intravenously, intraarterially, ntralesionally, intranasally, intravaginally, intrarectally, topically, intratumorally, systemically, intravesicularly, mucosally, orally, locally, via inhalation (e.g., aerosol inhalation), via injection, via infusion, via continuous infusion, via localized perfusion bathing target cells directly, via a catheter, via a lavage, in cremes, in lipid compositions (e.g., liposomes), or by other method or any combination of the foregoing as would be known to one of ordinary skill in the art (see, for example, Remington's Pharmaceutical Sciences, 1990). In some aspects, administration comprises oral administration.

The composition may be administered to (or taken by) the patient 1, 2, 3, 4, 5, 6, 7, 8, 9, 10, 11, 12, 13, 14, 15, 16, 17, 18, 19, 20 or more times, or any range derivable therein, and they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months, or 1, 2, 3, 4, 5, 6, 7, 8, 9, or 10 years, or longer, or any range derivable therein. It is specifically contemplated that the composition may be administered once daily, twice daily, three times daily, four times daily, five times daily, or six times daily (or any range derivable therein) and/or as needed to the patient. Alternatively, the composition may be administered every 2, 4, 6, 8, 12 or 24 hours (or any range derivable therein) to or by the patient. It is specifically contemplated that the composition may be administered daily over the course of multiple years.

The administration of the composition may precede or follow the PPI therapy by intervals ranging from minutes to weeks. In embodiments where the composition and PPI therapy are administered separately, one would generally ensure that a significant period of time did not expire between the time of each delivery, such that the therapeutic composition would still be able to exert an advantageously combined effect on the subject. In some embodiments, it is contemplated that one may administer both modalities within about 4-24 hours of each other or within about 4-12 hours of each other. In some embodiments, the PPI is administered before the composition. In some embodiments, the composition is administered before the PPI therapy. In some embodiments, the two treatments are concurrent.

Administration of the compositions of the disclosure to a patient/subject will follow general protocols for the administration of such compounds, taking into account the toxicity, if any, of the composition. It is expected that the treatment cycles would be repeated as necessary. It is also contemplated that various standard therapies may be applied in combination with the described therapy.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Effective amount” or “therapeutically effective amount” or “pharmaceutically effective amount” means that amount which, when administered to a subject or patient for treating a disease, is sufficient to effect such treatment for the disease. In some embodiments, the subject is administered at least about 1, 2, 3, 4, 5, 6, 7, 8, 9, 10, 20, 30, 40, 50, 60, 70, 80, 90, 100, 110, 120, 130, 140, 150, 160, 170, 180, 190, or 200 mg/kg or mg/kg/dose or more or any range derivable therein.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject of patient which may be at risk and/or predisposed to the disease but does not yet experience or display any or all of the pathology or symptomatology of the disease. Any measurable slowing of the rate of onset is encompassed by preventing.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 compares serum calcium 4 hours after taking a morning dose of placebo or EffCaMgCit (post-load). Individual data from 22 healthy subjects are shown after 2 weeks and 4 weeks of treatment with placebo or with EffCaMgCit while on esomeprazole. Closed squares with vertical bars indicate mean±SD for each treatment. Serum calcium was significantly higher on EffCaMgCit than on placebo at both 2 weeks and 4 weeks.

FIG. 2 shows the percentage of subjects with serum calcium less than 9 mg/dL (prevalence of abnormal serum calcium) after 2 weeks and 4 weeks of treatment with placebo or EffCaMgCit while taking esomeprazole. Serum calcium less than 8.6 mg/dL represents hypocalcemia; marginal hypocalcemia was defined as serum calcium≥8.6 mg/dL to <9.0 mg/dL. The dashed horizontal line indicates the prevalence value at baseline before taking any drug. The p value indicates significance of the difference between EffCaMgCit and Placebo phases. Hypocalcemia or marginal hypocalcemia was detected in 5 subjects at 2 weeks and in 4 subjects after 4 weeks on placebo (on esomeprazole alone). No subjects had hypocalcemia or marginal hypocalcemia on EffCaMgCit (plus esomeprazole).

FIG. 3 depicts serum PTH 4 hours after taking a morning dose of placebo or EffCaMgCit. Individual data from 22 healthy subjects are shown after 2 weeks and 4 weeks of treatment with placebo (plus esomeprazole) or EffCaMgCit (plus esomeprazole). Serum PTH was significantly lower on EffCaMgCit than on placebo at both 2 weeks and 4 weeks.

FIG. 4 compares the percentage of subjects with serum PTH>65 pg/ml (upper normal limit) after 2 weeks and 4 weeks of treatment with placebo (plus esomeprazole) versus EffCaMgCit (plus esomeprazole). The prevalence of high serum PTH rose from baseline before treatment (dashed horizontal line) to 2 weeks and 4 weeks on placebo while taking esomeprazole alone. Prevalence of high serum PTH was much lower on EffCaMgCit (plus esomeprazole) than on placebo (plus esomeprazole) at both 2 weeks and 4 weeks.

FIG. 5 compares serum CTX 4 hours after taking a morning dose of placebo or EffCaMgCit. Individual data from 22 healthy subjects are depicted after 2 weeks and 4 weeks of treatment with placebo (plus esomeprazole) or EffCaMgCit (plus esomeprazole). Serum CTX was significantly lower on EffCaMgCit than on placebo at both 2 weeks and 4 weeks.

FIG. 6 compares the percentage of subjects with serum CTX>0.63 pg/ml (upper normal limit) after 2 weeks and 4 weeks of treatment with placebo (plus esomeprazole) versus EffCaMgCit (plus esomeprazole). Six subjects had high serum CTX at both 2 weeks and 4 weeks on placebo. On EffCaMgCit, high serum CTX was found in no subjects at 2 weeks and in only 1 subject at 4 weeks.

FIG. 7 compares serum magnesium 4 hours after taking a morning dose of placebo or EffCaMgCit along with esomeprazole from the multi-dosing trial. Individual data from 22 healthy subjects are depicted after 2 weeks and 4 weeks of treatment with placebo or EffCaMgCit while on esomeprazole. Serum magnesium was significantly higher on EffCaMgCit than on placebo at both 2 weeks and 4 weeks. Data from the same subjects are connected by lines.

FIG. 8 compares the percentage of subjects with serum magnesium<2 mg/dL after 2 weeks and 4 weeks of treatment with placebo (plus esomeprazole) versus EffCaMgCit (plus esomeprazole). Hypomagnesemia is defined as serum magnesium<1.9 mg/dL. Marginal hypomagnesemia is defined as serum magnesium≥1.9 mg/dL and <2 mg/dL. Prevalence of hypomagnesemia or marginal hypomagnesemia rose from 2 subjects at baseline before treatment (dashed horizontal line) to 3 subjects at 2 weeks and 5 subjects at 4 weeks on esomeprazole alone with placebo. On EffCaMgCit with esomeprazole, no subjects had hypomagnesemia or marginal hypomagnesemia at both 2 weeks and 4 weeks.

FIG. 9 shows FEMg from the multidosing study in 22 subjects. Group means at Baseline, and at 2-4 weeks of treatment are compared between PPI alone (with placebo) and EffCaMgCit given with PPI. Circles and vertical lines indicate mean±SD. Significant difference between the two groups is shown by p values. Compared with placebo, EffCaMgCit yielded a significantly higher FEMg at both 2 and 4 weeks.

FIG. 10 illustrates 24-h urinary citrate at Baseline, at 2-4 weeks of treatment with esomeprazole+placebo, and at 2-4 weeks of treatment with esomeprazole+EffCaMgCit. Group means from the completed multidosing trial in 22 subjects are shown. *p<0.05 vs Baseline. Compared to Baseline, urinary citrate was significantly lower on PPI and Placebo, and significantly higher on PPI and EffCaMgCit.

FIG. 11 shows urinary ammonium at Baseline, at 2-4 weeks on esomeprazole+Placebo, and at 2-4 weeks on esomeprazole+EffCaMgCit. Group means from the from the completed multidosing trial in 22 subjects are depicted. **p<0.01 from Baseline; (†) p<0.001 between PPI+Placebo vs. PPI+EffCaMgCit. p between Baseline and PPI+Placebo was 0.18. Urinary ammonium on EffCaMgCit plus PPI was significantly lower from Baseline and from Placebo plus PPI.

FIG. 12 displays serum FGF23 4 hours after taking a morning dose of placebo or EffCaMgCit while on esomeprazole from the multi-dosing study. For each treatment, the group means at Baseline, at 2 weeks and at 4 weeks of treatment are compared between esomeprazole (+placebo) and esomeprazole (+EffCaMgCit). Circles and vertical lines indicate mean±SD. Difference between phases was significant at 4 weeks (p=0.0030) but not at Baseline and 2 weeks. There was a trend toward a decline from Baseline to 4 weeks of treatment for placebo=esomeprazole (p=0.1).

### DETAILED DESCRIPTION OF THE INVENTION

The inventors found that while on a high dose of PPI, EffCaMgCit (a) reduced serum CTX, a bone resorption marker, (b) raised serum and urinary Mg, (c) increased FEMg, (d) increased urinary citrate and decreased urinary ammonium, and (d) lowered putative serum FGF23. PPI alone showed changes in the opposite direction or not at all.

Notably, the percentage of subjects with abnormal serum calcium, PTH, CTX or magnesium was substantially lower on EffCaMgCit compared with placebo during PPI treatment. Some subjects had abnormal serum biochemistry at baseline (before beginning esomeprazole). In some subjects, abnormal serum biochemistry developed while on placebo (esomeprazole alone). The foregoing results suggested that EffCaMgCit prevents/treats hypocalcemia, secondary hyperparathyroidism, high bone resorption, as well as hypomagnesemia from PPI therapy. Thus, the inventors hypothesize that EffCaMgCit suppresses parathyroid function by improving calcium absorption and raising serum calcium. Suppressed parathyroid function would then reduce osteoclastic resorption and prevent/treat bone loss that would eventually lead to fragility fractures. By improving magnesium absorption, EffCaMgCit would raise serum magnesium and prevent/treat hypomagnesemia that would lead to magnesium deficiency. Moreover, EffCaMgCit overcame tendency toward acid load from PPI. Combined with other effects described above, this action contributed toward reduced risk of CKD by EffCaMgCit.

### A. IMPACT OF PROTON PUMP INHIBITORS

PPIs have significant side effects that can cause serious issues for patients that take them for many years. Despite potential usefulness of calcium-magnesium supplements to overcome these side effects, the available preparations lack adequate solubility and bioavailability. Because of this, there is a need for a calcium-magnesium preparation that delivers soluble calcium and magnesium during treatment with a PPI.

Solubility of calcium and magnesium salts are dependent on pH (Pak, 1989). PPIs inhibit gastric acid secretion. By increasing luminal pH, PPIs can impair the solubility of calcium and magnesium salts. The resulting impairment in calcium and magnesium absorption might lead to parathyroid stimulation and bone loss as well as hypomagnesemia.

PPI impairs the solubility and hence the absorbability of calcium and magnesium salts by reducing gastric acid secretion. Stimulation of duodenal bicarbonate secretion by PPI (Mertz-Nielson, 1996) further impairs solubility of these salts. PPI might also inhibit passive magnesium absorption (Thongon, 2011). These inhibitory effects of PPI should be mitigated by the special composition of EffCaMgCit that delivers calcium and magnesium in an optimally bioavailable form.

1. Bone Loss

The FDA's Drug Safety Communication in 2011 cited six articles reporting on increased risk of skeletal fractures during PPI use (FDA, 2011). The risk was higher in older individuals, bigger doses of PPI and longer duration of treatment. In a meta-analysis of 11 studies, PPI use was associated with about 30% increase in hip fractures (Khalili, 2012). A plausible explanation for the skeletal complication of PPI is the induction of hypo- or achlorhydria that impairs the solubility and hence the bioavailability of calcium. The consequent stimulation of parathyroid function would cause bone loss leading eventually to fragility fractures.

CTX is a degradation product of bone collagen; it is released into circulation when bone is resorbed by osteoclasts. Serum CTX is an excellent biochemical marker for bone resorption. Serum CTX is elevated in clinical states of high bone turnover. It is reduced by drugs that reduce bone resorption. Serum CTX is used commonly to evaluate response to anti-osteoporosis drugs. Therefore, there is a need for a treatment that can lower serum CTX during treatment with a PPI.

The inventors found that at both 2 weeks and 4 weeks of treatment, serum CTX was significantly lower on EffCaMgCit (plus esomeprazole) compared with placebo (plus esomeprazole). Six subjects had high serum CTX at both 2 weeks and 4 weeks of placebo (on esomeprazole). On EffCaMgCit (plus esomeprazole), high serum CTX was found in none of the subjects at 2 weeks and only one subject at 4 weeks.

2. Hypomagnesemia

In FDA's Adverse Event Reporting System in 2012, the odds ratio of hypomagnesemia was 2.76 between omeprazole use and non-omeprazole use (Tamura, 2012). The risk increased with longer duration of PPI use. There is concern over this complication, since hypomagnesemia has been associated with increased cardiovascular death (Sakaguchi, 2014) and deterioration of renal function (van Laecke, 2013). Thus, there is a need for a treatment that can increase serum and urinary magnesium during treatment with a PPI.

Common magnesium salts require gastric acid for full solubility. Thus, a plausible explanation for the development of hypomagnesemia from PPI use is the induction of hypo- or achlorhydria that impairs the solubility and hence the bioavailability of magnesium salts (Kuipers, 1999). PPI might also inhibit magnesium absorption by impairing passive absorption of magnesium (Thongon, 2011) or by promoting bicarbonate secretion (Mertz-Nielson, 1996).

Hypomagnesemia is an insufficient gauge of magnesium deficiency, which reflects depletion of magnesium stores in tissues. It is magnesium deficiency that is responsible for neuromuscular disturbances, arrhythmia, cardiovascular death (Sakaguchi, 2014) and deterioration of renal function (van Laecke, 2013; Xie, 2017). Serum magnesium may be normal in some cases of magnesium deficiency. Free muscle magnesium might be an excellent marker of intracellular magnesium deficiency (Irish, 1997). However, free muscle magnesium is hard to procure because of the need for costly and cumbersome magnetic resonance spectrophotometry.

FEMg is a good biomarker of systemic Mg deficiency as it exploits the renal response to defend body Mg deficit. In addition, FEMg helps distinguish renal versus extrarenal losses of magnesium (Elisaf, 1997), although the likelihood of PPI inducing renal Mg wasting is very low. Inconsistent response to magnesium supplementation indicates that hypomagesemia/magnesium deficiency of PPI use is most likely intestinal in origin, reinforcing the usefulness of FEMg. The inventors disclose here new evidence that FEMg is a useful biological gauge in quantitating the effect of EffCaMgCit on magnesium deficiency.

The inventors found that serum magnesium was significantly higher after EffCaMgCit than after placebo. At baseline (before starting esomeprazole), 9% of subjects had hypomagnesemia or marginal hypomagnesemia (<2 mg/dL). During treatment with placebo with esomeprazole, hypomagnesemia or marginal hypomagnesemia was found in a higher percentage of subjects (14% at 2 weeks, and 23% at 4 weeks). During treatment with EffCaMgCit with esomeprazole, no subjects had hypomagnesemia or marginal hypomagnesemia. The inventors also found that FEMg tended to decline on 4 weeks of PPI alone, but increased substantially when EffCaMgCit was co-administered with PPI.

3. Chronic Kidney Disease

PPI use has been associated with increased incidence of CKD from a meta-analysis of published trials (Wijarnpreecha, 2017) and a large VA database (Xie, 2017). The exact cause of this renal complication remains elusive. However, the inventors' own data suggests a causal role of hypomagnesemia/magnesium deficiency, acid load, and FGF23 excess from PPI for the increased risk of CKD. Therefore, there is a need for a treatment that can increase serum and urinary magnesium, raise urinary citrate, decrease urinary ammonium, and lower FGF23 during treatment with a PPI. All these factors were correctible by EffCaMgCit.

Hypomagnesemia alone has been associated with impaired renal function (Sakaguchi, 2015; van Laecke, 2013). In patients on hemodialysis (Stage 5D), those on PPI have a reduced survival rate (Ago, 2016). Excessive acid load is associated with renal impairment (Banerjee, 2015), and progression of CKD slows while on alkali therapy (Phisitkul, 2010; de Brito-Ashurst, 2009; Mahaj an, 2010). Previous evidence has led to the common belief that PPI does not deliver an acid load, since the reduced proton secretion in the upper bowel by PPI is compensated by a commensurate fall in bicarbonate secretion by the pancreas (Cameron, 2012). However, a contrary evidence has appeared, showing that PPI promotes duodenal bicarbonate secretion (Mertz-Nielson, 1996). Thus, patients with CKD Stage 5D on PPI with hypomagnesemia might be particularly amenable to EffCaMgCit therapy.

The inventors' surprisingly and unexpectedly found that PPI might deliver an acid load. The combination of both hypomagnesemia/magnesium deficiency and delivery of an acid load might contribute to renal impairment. Increased ammoniagenesis from acid load might also contribute to renal tubular injury by interacting with complement (Nath, 1985). The inventors determined that a delivery of acid load by PPI was overwhelmed by alkali load from EffCaMgCit. Correction of magnesium deficiency, acid load, and enhanced ammoniagenesis by EffCaMgCit should avert incident CKD.

The inventors found that in CKD Stage 3, serum magnesium rose significantly but only slightly following EffCaMgCit treatment; it did not change after calcium acetate (data not shown here). The absorbed Mg from EffCaMgCit was largely eliminated into urine in subjects with residual renal function. In Stage 5D without residual renal function, however, EffCaMgCit produced a striking increase in serum Mg, whereas calcium acetate did not.

Serum FGF23 is elevated in CKD and is believed to be nephrotoxic as well as cardiotoxic. A reduced dietary acid load was recently shown to be associated with a lower incidence of end stage renal disease (Banerjee, 2015). The inventors found that serum FGF23 tended to rise from Baseline on PPI alone, but it tended to decline when EffCaMgCit was added. The cause for the stimulated synthesis of FGF23 by PPI is unknown; it might due to the acid load (Krieger, 2012) or magnesium deficiency (van Angelen, 2013). The reduction in serum FGF23 by EffCaMgCit is probably the result of alkali load, magnesium repletion, and intestinal binding of phosphate by EffCaMgCit.

### B. EFFERVESCENT CALCIUM MAGNESIUM CITRATE

In some aspects, the present invention relates to a method of administering a composition to the patient that comprises calcium, magnesium, and citrate (EffCaMgCit). In some embodiments, the composition is intended to be dissolved in water before oral ingestion. To obtain a customary, clinically effective dose of calcium (from 250 mg to 600 mg per unit dose) and magnesium (from 70 mg to 175 mg per unit dose), EffCaMgCit contains at least 10 meq of citrate excess and at least 20 meq of total soluble citrate per unit dose, to yield Ca/citrate molar ratio equal to or less than 0.7. In particular embodiments, EffCaMgCit contains 380 mg calcium per unit dose, 122 mg magnesium per unit dose, 21 meq citrate excess and total citrate of 50 meq per unit dose, and Ca/citrate molar ratio of 0.57.

To make EffCaMgCit, a solid powder preparation of poorly soluble calcium carbonate is added to water, along with magnesium citrate and citric acid. This mixture elaborates carbon dioxide gas as it rapidly dissolves; thus, the poorly soluble calcium carbonate is rendered readily soluble. The use of a powder mixture of calcium, magnesium and citrate that dissolves rapidly and fully, permitting ingestion as a liquid is beneficial. For example, some patients prefer to drink a liquid preparation rather than swallow tablets. The rapidity with which the powder formulations dissolve overcomes the problem of somewhat poor solubility of tablet formulations.

EffCaMgCit contains additional citric acid to yield a calcium/citrate molar ratio of ˜0.6. Calcium supplements in the marketplace have a much higher Ca/citrate molar ratio, with a value of infinity for calcium carbonate and calcium phosphate, and 1.5 for calcium citrate. EffCaMgCit also has a greater citrate excess (21 meq per unit dose) than conventional preparations. These properties provide rapid and complete solubility of the composition.

Moreover, EffCaMgCit has citrate excess or free citrate of 21 meq citrate per unit dose (total citrate minus total calcium+magnesium; 50−19−10=21). The total soluble citrate from a single dose of EffCaMgCit is 50 meq (=10 meq from magnesium citrate and 40 meq from citric acid). Since EffCaMgCit is completely solubilized in water before ingestion, all of its content of citrate contributes to total soluble citrate. That is not the case with tablet formulations of calcium citrate that are partially soluble. Furthermore, since EffCaMgCit contains 50 meq of total soluble citrate per unit dose and 21 meq of free citrate, it can elicit a rise in serum citrate comparable or superior to that of 40 meq citric acid.

The composition of EffCaMgCit possesses not only a low Ca/citrate ratio but also contains sufficient free citrate and total free citrate. A low Ca/citrate molar ratio alone does not ensure efficient solubility or satisfactory citratemic response (rise in serum citrate), unless it is accompanied by adequate citrate excess. The amount of free and total soluble citrate contained in EffCaMgCit is designed to attenuate the expected rise in serum ionized calcium from the unit dose of calcium 380 mg. When a higher unit dose of calcium is to be used, the amount of free citrate and total soluble citrate must be adjusted to maintain the Ca/citrate molar ratio low.

Based on the needs of average healthy individuals, it is believed that the optimal amounts per unit dose of EffCaMgCit are: (a) calcium 380 mg, to provide optimum calcium bioavailability without provoking hypercalcemia, (b) magnesium 122 mg, to offer bioavailable magnesium without provoking diarrhea, (c) Ca/citrate molar ratio of ˜0.6, (d) citrate excess of ˜20 meq, and (e) total soluble citrate of 50 meq per unit dose. However, other dosages may be effective. For example, in persons with defective intestinal calcium absorption, a higher amount of calcium might be desired. This would necessitate appropriate changes in free citrate and total soluble citrate. Alternatively, in patients with absorptive hypercalciuria suffering from kidney stones due to high intestinal calcium absorption, a lower unit dose of calcium might be preferred, while maintaining the same citrate excess and total soluble citrate. Persons skilled in the art might modify the amounts of various components of the composition, depending on circumstances, individual needs and conditions.

### C. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Multi-Dosing Study

The inventors conducted a multi-dosing trial, in which the effect of EffCaMgCit was compared with placebo in a crossover trial in 22 healthy subjects taking PPI at twice the usual dose (esomeprazole 40 mg/day) for 28 days, representing the recommended treatment format for erosive esophagitis-gastritis. During the EffCaMgCit phase, subjects took EffCaMgCit containing 19 meq calcium and 10 meq magnesium twice per day for 28 days. During the Placebo Phase, the subjects took placebo for 28 days. During both phases, the subjects took esomeprazole 40 mg per day. Blood samples were obtained 4 hours after EffCaMgCit dose (in multi-dosing trials with esomeprazole), rather than in the fasting state. Tamsen: no study with PPI in CKD. Peak absorption of calcium and magnesium occurs 2-4 hours after oral load. Thus, pharmacokinetic and pharmacodynamic effects of EffCaMgCit can best be captured in post-load samples.

### Example 2

### Effect of EffCaMgCit on Serum CTX

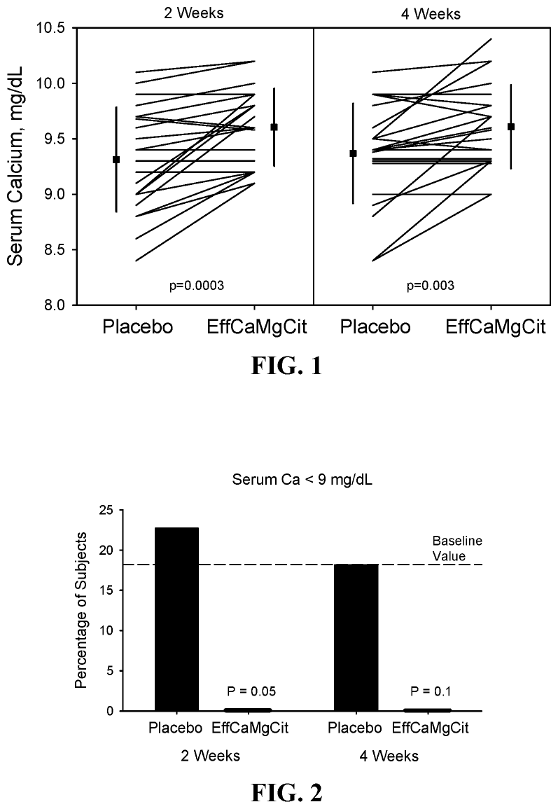

A venous blood sample was obtained at 4 hours after the morning dose of EffCaMgCit or placebo, for the analysis of Ca, PTH, CTX and Mg after 2 weeks and 4 weeks of treatment. Serum calcium was significantly higher after EffCaMgCit than placebo, with virtually every subject showing a higher value. In the Placebo group (while on esomeprazole), a low or marginally low serum calcium (<9 mg/dL) was encountered in 23% of subjects at 2 weeks and in 18% of subjects at 4 weeks. After EffCaMgCit (with esomeprazole), none of the patients had low or marginally low serum calcium. Thus, not only did EffCaMgCit raise serum calcium, but it also corrected low or marginally low serum calcium. At two weeks of placebo (esomeprazole alone), a higher percentage of subjects had low or marginally low serum calcium than at baseline before esomeprazole (23% vs. 18%). Thus, a low or marginally low serum calcium developed during two weeks of esomeprazole therapy in an additional patient. This subject had normal serum calcium on EffCaMgCit (plus esomeprazole). FIGS. 1 and 2.

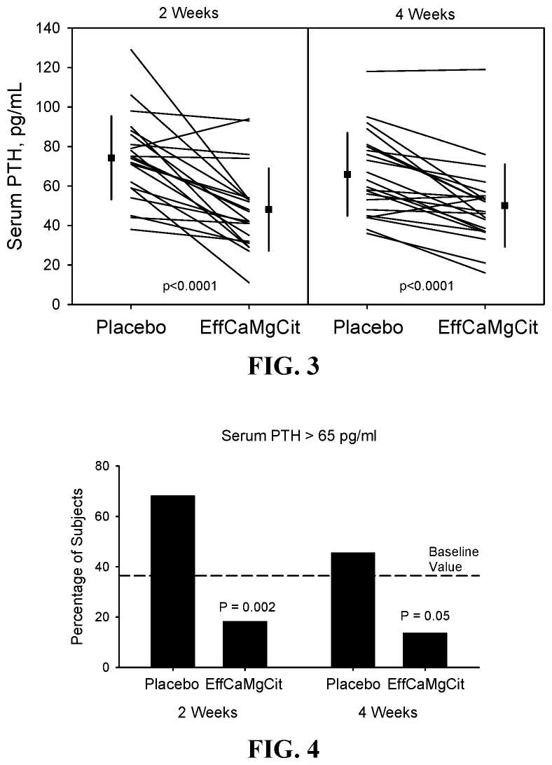

In the same multi-dosing trial, serum PTH was significantly lower after 2 weeks and 4 weeks of treatment with EffCaMgCit (plus esomeprazole) compared with placebo (plus esomeprazole). Prevalence of high serum PTH (>65 pg/ml) was much lower during 2 weeks and 4 weeks of treatment with EffCaMgCit (plus esomeprazole) compared with placebo (plus esomeprazole). At baseline (before esomeprazole treatment), 8 subjects had high serum PTH. While on placebo (esomeprazole alone), a higher number of subjects had high serum PTH at 2 weeks (n=15) and 4 weeks (n=10), indicating that some subjects acquired secondary hyperparathyroidism on esomeprazole. On EffCaMgCit (plus esomeprazole), fewer subjects had high serum PTH at 2 weeks (n=4) and at 4 weeks (n=3). FIGS. 3 and 4.

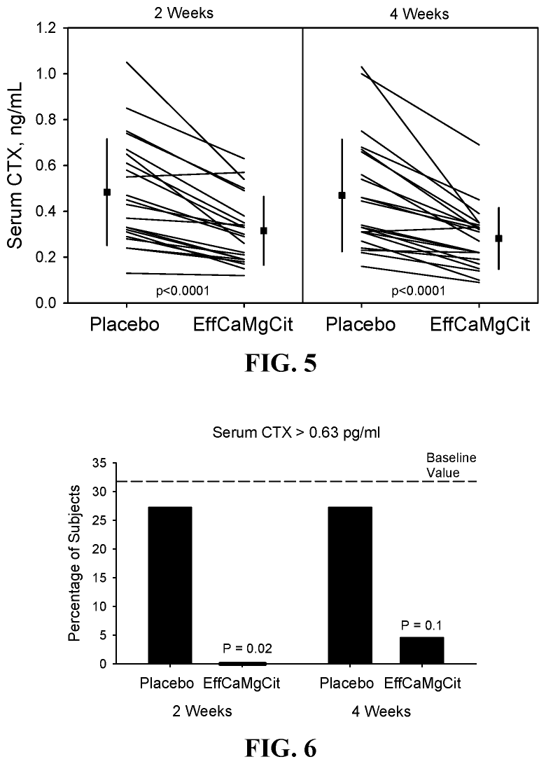

At both 2 weeks and 4 weeks of treatment, serum CTX was significantly lower on EffCaMgCit (plus esomeprazole) compared with placebo (plus esomeprazole). Six subjects had high serum CTX at both 2 weeks and 4 weeks of placebo (on esomeprazole). On EffCaMgCit (plus esomeprazole), high serum CTX was found in none of the subjects at 2 weeks and only one subject at 4 weeks. FIGS. 5 and 6.

### Example 3

### Effect of EffCaMgCit on Serum Magnesium

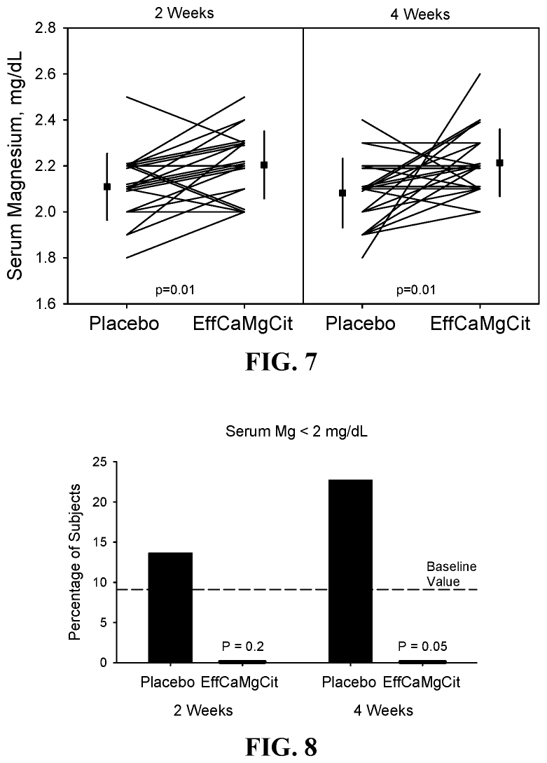

Serum magnesium was determined at 2 weeks and 4 weeks at 4 hours after the morning dose. Serum magnesium was significantly higher after EffCaMgCit than after placebo. At baseline (before starting esomeprazole), 9% of subjects had hypomagnesemia or marginal hypomagnesemia (<2 mg/dL). During treatment with placebo with esomeprazole, hypomagnesemia or marginal hypomagnesemia was found in a higher percentage of subjects (14% at 2 weeks, and 23% at 4 weeks). During treatment with EffCaMgCit with esomeprazole, no subjects had hypomagnesemia or marginal hypomagnesemia. FIGS. 7 and 8.

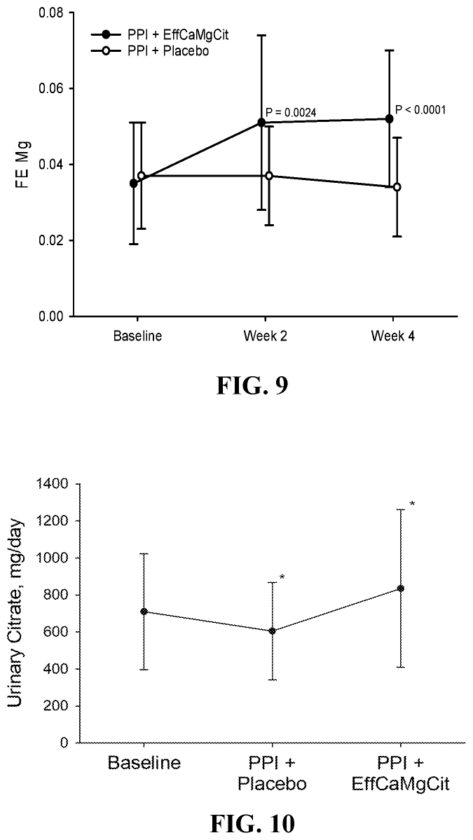

The inventors also found that FEMg trended downward on PPI alone, but increased prominently when EffCaMgCit was added. FIG. 9. The inventors have initiated a trial wherein PPI and EffCaMgCit treatment will be monitored by the measurement of FEMg and free muscle magnesium, as well as serum magnesium.

### Example 4

### Effect of EffCaMgCit on Acid Load and Serum FGF23

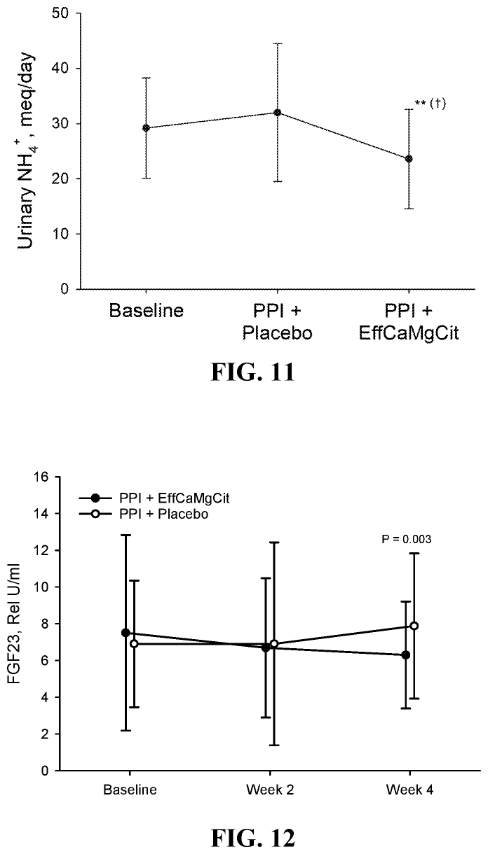

24-h urinary citrate and ammonium, urinary markers of acid-base balance, were determined at baseline and after 2-4 weeks of treatment with esomeprazole (plus placebo) and after treatment with esomeprazole (plus EffCaMgCit). On PPI alone (with placebo), urinary citrate was significantly lower than at baseline. Urinary ammonium was marginally higher than at Baseline. On esomeprazole with EffCaMgCit, urinary citrate was significantly higher and urinary ammonium was significantly lower than at Baseline. These results indicate that delivery of an acid load by PPI was overwhelmed by alkali load from EffCaMgCit. FIGS. 10 and 11.

Serum FGF23 is elevated in CKD and is believed to be nephrotoxic as well as cardiotoxic. In the study, serum FGF23 tended to rise from Baseline on PPI alone, but it tended to decline when EffCaMgCit was added. The difference between the two groups was significant at 4 weeks of treatment but not at Baseline. FIG. 12.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Ago et al., Hemodial., Int., 20(4): 580-588, 2016.
Banerjee et al., J. Am. Soc. Nephrol., 26: 15. doi: 10.1681/ASN.2014040332, 2015.
Cameron et al., Kidney Int., 81(11): 1123-30, 2012.
de Brito-Ashurst et al., J Am. Soc. Nephrol., 20: 2075-2084, 2009.
Elisaf M et al., Magnes. Res., 10(4): 315-20, 1997.
FDA FDA drug safety communications: Mar. 2, 2011.
Irish et al., Nephron, 76: 20-25, 1997.
Khalili et al., Brit. Med. 1, 344: e372, 2012.
Kuipers et al., Netherl. J. Med., 67: 169-172, 2009.
Irish et al., Nephron, 76: 20-25, 1997.
Krieger et al., Am. J. Ren. Physiol., 303: F421-F436, 2012.
Mahajan et al., Kidney Int., 78: 303-309, 2010.
Mertz-Nielsen et al., Gut, 38(1): 6-10, 1996.
Nath et al., J. Clin. Invest., 76: 667-675, 1985.
Phisitkul et al., Kidney Int., 77: 617-623, 2010.
Pak et al., J. Bone Min. Res., 4:119-127, 1989.
Roux et al., Calcif. Tissue Int., 84:13-19, 2009.
Sakaguchi et al., PLOS ONE|DOI:10.1371/journal.poGne.0116273, 2014.
Tamura et al., Int. J. Med. Sci., 9: 322-326, 2012.
Thongon et al., World J. Gastroenterol., 17(12): 1574-83, 2011.
Thongon et al., Exp. Molec. Med., 44: 684-693, 2012.
Van Angelen et al., Nephrol. Dial. Transplant, 28(12): 2983-2993, 2013.
van Laecke et al., Am. J. Med., 126: 825-831, 2013.
Wijarnpreecha et al., Dig. Dis. Sci., 62(10): 2821-2827, 2017.
Xie et al., Kidney Int., 91(6): 1482-1494, 2017.

What is claimed is:

1. A method of reducing serum C-carboxy-terminal telopeptide (CTX) in a patient receiving or prescribed a proton pump inhibitor (PPI) therapy, the method comprising administrating a composition comprising from about 250 mg calcium to about 600 mg of calcium, from about 70 mg magnesium to about 175 mg of magnesium, at least 10 meq of citrate excess, and at least 20 meq of total soluble citrate, wherein the Ca/citrate molar ratio is from 0.4 to 0.7, wherein serum C-carboxy-terminal telopeptide (CTX) is reduced.

2. The method of claim 1, wherein the composition comprises 380 mg (19 meq) of calcium, 122 mg (10 meq) of magnesium, 21 meq of citrate excess, and 50 meq of total soluble citrate, wherein the Ca/citrate molar ratio is 0.57.

3. The method of claim 1, wherein bone loss is decreased.

4. A method of claim 1, wherein the mixture is in the form of a powder, to be dissolved in aqueous medium before taking orally.

5. A method of claim 1, wherein said administration comprises oral administration.

6. The method of claim 1, wherein the composition is further defined as an aqueous solution.

7. The method of claim 1, further comprising preparing the aqueous solution by dissolving the dose comprising the mixture in water.

8. The method of claim 1, wherein the composition is mixed with a PPI.

9. The method of claim 1, wherein the patient is a human.

10. A method of increasing serum and urinary Mg in a patient receiving or prescribed a proton pump inhibitor (PPI) therapy, the method comprising administrating a composition comprising from about 250 mg calcium to about 600 mg of calcium, from about 70 mg magnesium to about 175 mg of magnesium, at least 10 meq of citrate excess, and at least 20 meq of total soluble citrate, wherein the Ca/citrate molar ratio is from 0.4 to 0.7, wherein the serum and urinary Mg is increased.

11. The method of claim 10, wherein the composition comprises 380 mg (19 meq) of calcium, 122 mg (10 meq) of magnesium, 21 meq of citrate excess, and 50 meq of total soluble citrate, wherein the Ca/citrate molar ratio is 0.57.

12. The method of claim 11, wherein tissue magnesium stores are increased.

13. The method of claim 10, wherein the patient has or is at risk of having CKD.

14. A method of increasing FEMg in a patient receiving or prescribed a proton pump inhibitor (PPI) therapy, the method comprising administrating a composition comprising from about 250 mg calcium to about 600 mg of calcium, from about 70 mg magnesium to about 175 mg of magnesium, at least 10 meq of citrate excess, and at least 20 meq of total soluble citrate, wherein the Ca/citrate molar ratio is from 0.4 to 0.7, wherein the FEMg is increased.

15. The method of claim 14, wherein the composition comprises 380 mg (19 meq) of calcium, 122 mg (10 meq) of magnesium, 21 meq of citrate excess, and 50 meq of total soluble citrate, wherein the Ca/citrate molar ratio is 0.57.

16. The method of claim 14, wherein the patient has or is at risk of having CKD.

17. A method of increasing urinary citrate and reducing urinary ammonium in a patient receiving or prescribed a proton pump inhibitor (PPI) therapy, the method comprising administrating a composition comprising from about 250 mg calcium to about 600 mg of calcium, from about 70 mg magnesium to about 175 mg of magnesium, at least 10 meq of citrate excess, and at least 20 meq of total soluble citrate, wherein the Ca/citrate molar ratio is from 0.4 to 0.7, wherein the urinary citrate is increased and urinary ammonium is decreased.

18. The method of claim 17, wherein the composition comprises 380 mg (19 meq) of calcium, 122 mg (10 meq) of magnesium, 21 meq of citrate excess, and 50 meq of total soluble citrate, wherein the Ca/citrate molar ratio is 0.57.

19. The method of claim 17, wherein the acid load is increased in the patient.

20. The method of claim 17, wherein the patient has or is at risk of having CKD.

21. A method of decreasing putative serum fibroblast growth factor 23 (FGF23) in a patient receiving or prescribed a proton pump inhibitor (PPI) therapy, the method comprising administrating a composition comprising from about 250 mg calcium to about 600 mg of calcium, from about 70 mg magnesium to about 175 mg of magnesium, at least 10 meq of citrate excess, and at least 20 meq of total soluble citrate, wherein the Ca/citrate molar ratio is from 0.4 to 0.7, wherein the serum FGF23 is decreased.

22. The method of claim 21, wherein the composition comprises 380 mg (19 meq) of calcium, 122 mg (10 meq) of magnesium, 21 meq of citrate excess, and 50 meq of total soluble citrate, wherein the Ca/citrate molar ratio is from 0.57.

23. The method of claim 21, wherein the patient has or is at risk of having CKD.

24. A method of treating excessive bone loss and hypomagnesemia-magnesium deficiency and of preventing increased risk of CKD during prolonged PPI treatment, the method comprising administrating a composition comprising from about 250 mg calcium to about 600 mg of calcium, from about 70 mg magnesium to about 175 mg of magnesium, at least 10 meq of citrate excess, and at least 20 meq of total soluble citrate, wherein the Ca/citrate molar ratio is from 0.4 to 0.7.
